SEQUENCE LISTING (1 sequence)

SEQ ID NO: 1 - DNA, 476 bp, Strawberry Vein Banding Virus (SVBV) Strain E3
aactatgctgatgacaagataattctaataagcaattattcagaattaatcaaggagaaagaattaataactctttcagaatatgaagcccgctttacaagtggccagctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaacc

(19) United States
(12) Patent Application Publication
(10) Pub. No.: US 2005/0003354 A1
(43) Pub. Date: Jan. 6, 2005

(54) Methods for improving or altering promoter/enhancer properties

(75) Inventors: Jack Wilkinson, Redwood City, CA (US); Kevin McBride, Davis, CA (US)

Correspondence Address: MAXYGEN, INC.;INTELLECTUAL PROPERTY DEPARTMENT, 515 GALVESTON DRIVE, RED WOOD CITY, CA, 94063, US

(73) Assignee: Maxygen, Inc.

(21) Appl. No.: 10/081,526

(22) Filed: Feb. 21, 2002

Related U.S. Application Data
(60) Provisional application No. 60/271,067, filed on Feb. 21, 2001.

(51) Int. Cl. C12Q 1/68; C07H 21/04; C12P 19/34; C12N 9/22

(52) U.S. Cl. 435/6; 435/69.1; 435/91.2; 435/199; 435/455; 435/320.1; 435/325; 536/23.2

(57) ABSTRACT

The present invention provides methods of reassembling polynucleotides and selecting polynucleotides with altered transcriptional regulatory activity.

### CROSS-REFERENCE TO RELATED APPLICATIONS

The present application claims benefit of priority from United States Patent Application Ser. No. (USSN) 60/271,067, filed Feb. 21, 2001, which is incorporated herein by reference in its entirety and for all purposes.

### FIELD OF THE INVENTION

This invention relates to methods for the facilitated evolution of transcriptional regulatory sequences.

### BACKGROUND OF THE INVENTION

Gene expression is controlled, to a large extent, by nucleotide sequences called promoters and enhancers that flank the coding region for a given protein. In some instances, these sequences also reside within exon and intron sequences of the gene. The nucleotide sequences comprising these regulatory elements, known as cis-acting sequences, serve as binding sites for protein factors that can facilitate or repress the transcription of the gene. In addition, these sequences may, either directly or indirectly through protein interactions, bind to the nuclear scaffold or adopt conformations that affect gene expression. It is the complex interaction between these nucleotide sequences and protein factors within each cell that determines the strength, timing, cell and tissue-specificity of each gene's expression.

In general, the promoter and enhancer sequences for a given gene across species, or for genes within a species with shared expression characteristics, are not as well conserved as protein coding regions. In fact, in many cases, it is difficult to identify any regions of extended homology between promoters of various genes. This is partly due to the fact that protein factors that interact with these sequences often bind to relatively small target regions in which significant heterogeneity is tolerated. Therefore, the selective pressure to maintain specific sequences in a specific order within a regulatory region is more relaxed than for protein coding regions. In addition, due to the flexibility of the DNA backbone, protein binding to the regulatory sequences can often occur in both orientations and at great distances from the transcription start site while maintaining the desired expression characteristics.

Studies have previously described the formation of synthetic promoters by assembly of cis-acting sequences. For example, Gelvin et al., U.S. Pat. No. 5,955,646 describes the formation of an active synthetic plant promoter from a combination of known cis-acting enhancer elements from the Agrobacterium octopine synthase (ocs) and mannopine synthase (mas) genes. Similarly, Li et al., Nat. Biotechnol. 17:241-245 (1999) describes the formation of synthetic promoters by combining known muscle-specific regulatory elements. Both references, however, only describe combining known, well-defined cis-acting elements. In contrast, there have been no reports of constructing promoter segments without regard to the presence or absence of regulatory elements. The present invention addresses this and other problems.

### BRIEF DESCRIPTION OF THE FIGURES

FIG. 1 is a schematic representation of single promoter fragmentation and re-assembly. The figure demonstrates that segments are assembled randomly and provides examples of how the segments can be re-assembled, i.e., inverted relative to other segments, multiple copies of the same segment, etc.

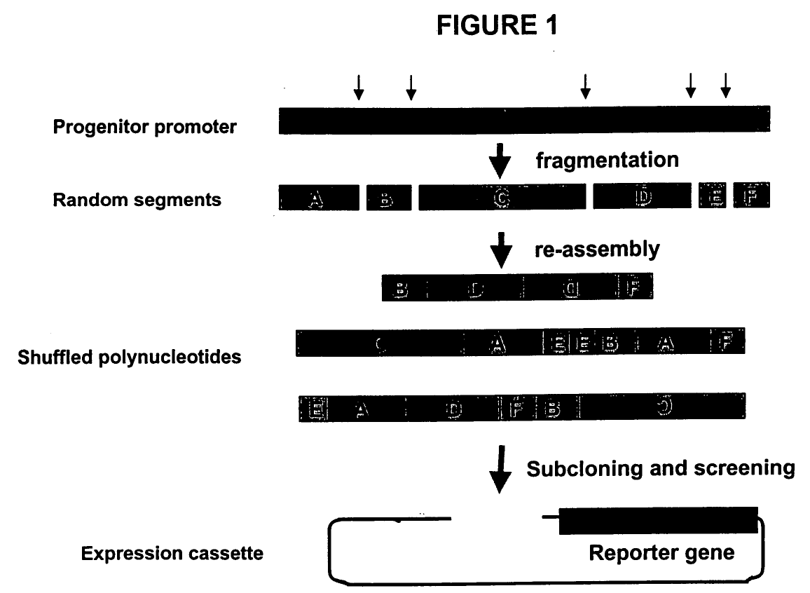

FIG. 2 is a schematic representation of multiple promoter fragmentation and re-assembly.

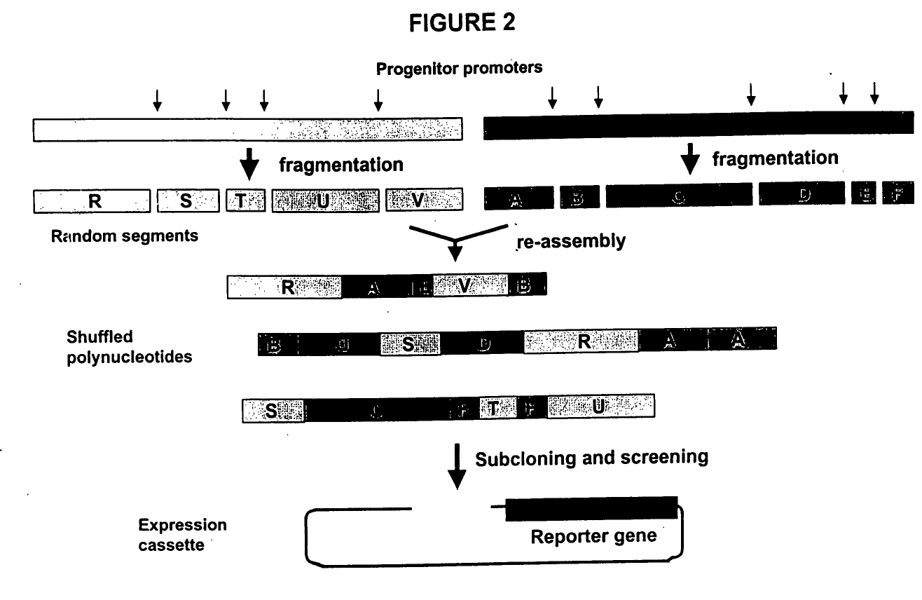

FIG. 3 is a schematic representation of single promoter fragmentation and re-assembly with oligonucleotide spiking.

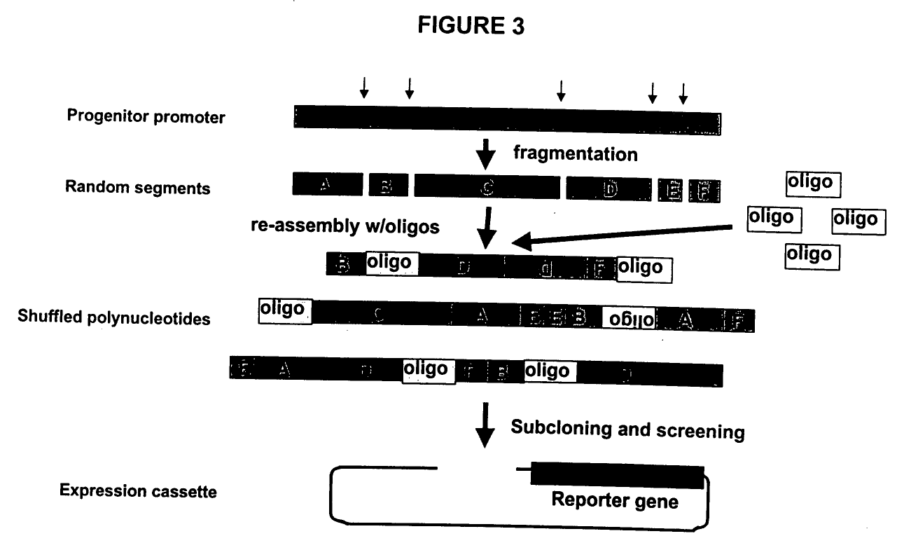

FIG. 4 is a schematic representation of fragmentation and re-assembly of mutated promoters.

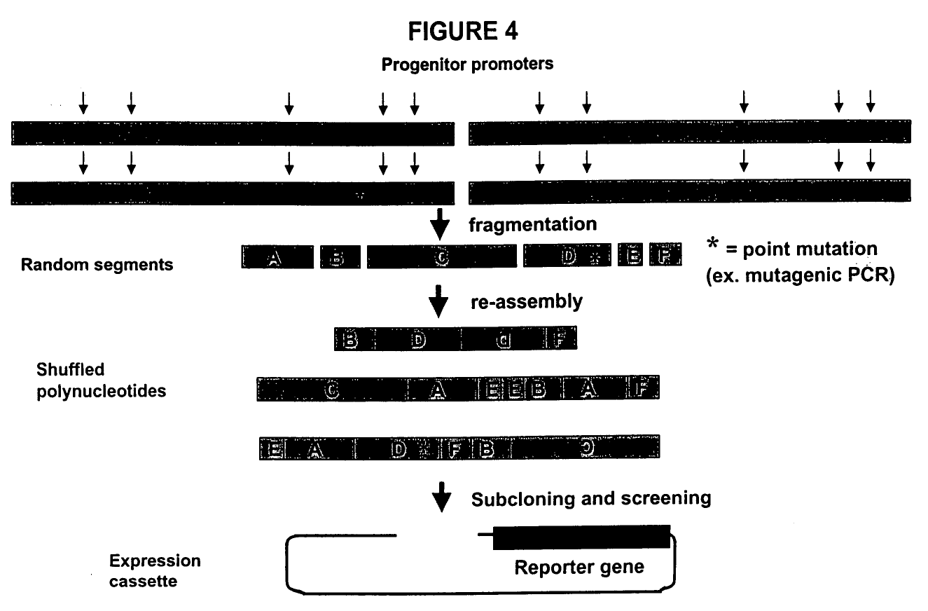

### SUMMARY OF THE INVENTION

This invention provides methods of reassembling polynucleotides involved in transcription. In some embodiments, the methods of the invention comprise 1) providing a plurality of random polynucleotide segments from one or more transcriptional regulatory progenitor polynucleotides; 2) assembling the plurality of segments in a random fashion, thereby forming a plurality of reassembled polynucleotides; and 3) selecting a reassembled polynucleotide with a different transcriptional regulatory activity than the progenitor polynucleotides.

In some embodiments, the segments are from 5 to 5,000 base pairs long. In some embodiments, the segments are less than 50 base pairs. In some embodiments, the segments are greater than 49 base pairs. In some embodiments, the ligated segments are size-selected by various means (e.g. gel fractionation and purification) to ensure that the assembled promoters or enhancers exceed a certain minimum length.

In some embodiments, the assembling stpp comprises ligating the segments. In some embodiments, the ligating step is performed with a DNA ligase or a topoisomerase. The methods of the invention provide for ligating segments of one or at least two distinct promoter or enhancer polynucleotides. In some embodiments, the random segments are obtained by random cleavage or random amplification of one or more transcriptional regulatory progenitor polynucleotides. The reassembled polynucleotide can comprise a promoter and/or an enhancer.

The selection step of the invention can comprise, for example, selecting reassembled polynucleotides with increased or decreased transcriptional activity relative to the transcriptional activity of a progenitor polynucleotide. Alternatively, or in addition, the reassembled polynucleotides can be selected on the basis of transcriptional activity in at least one cell or tissue type where the progenitor polynucleotide lacks activity. In other embodiments, the reassembled polynucleotides can be selected on the basis of lack of transcriptional activity in at least one cell or tissue type where the progenitor polynucleotide has activity. In some embodiments, the reassembled polynucleotides are selected on the basis of response to biotic or abiotic stimuli. In some embodiments, the reassembled polynucleotides are selected on the basis of transcriptional activity at a different developmental stage of an organism relative to the transcriptional activity of a progenitor polynucleotide. The selection step can be performed, for example, by ligating the reassembled polynucleotide to a reporter gene and measuring reporter gene activity.

In some embodiments, the segments are formed by nicking and subsequent end-repair of DNA that is altered by radiation, oxidation, or a chemical agent. In some embodiments, the segments are formed by cleaving one or more progenitor polynucleotides with a restriction endonuclease, DNaseI, or by mechanical cleavage. In some embodiments, the segments are formed by nicking and subsequent end-repair of DNA that is altered by radiation, oxidation, or a variety of chemical agents. In some embodiments, the segments are formed in a thermocyclic amplification reaction such as the polymerase chain reaction. In some embodiments, the plurality of segments comprise oligonucleotides. For example, the oligonucleotides can correspond to a transcription factor binding site. Alternatively, the nucleotide sequence of the oligonucleotides are not from a transcriptional regulatory polynucleotide.

The reassembled polynucleotide can be shorter or longer than the progenitor polynucleotide. In some embodiments, the progenitor polynucleotides comprise allelic variants of a transcriptional regulator polynucleotide, for example, plant, yeast, fungal, mammalian, viral and/or bacterial transcriptional regulatory polynucleotides. In some embodiments, the progenitor polynucleotides consist of one transcriptional regulatory polynucleotide. In other embodiments, the progenitor polynucleotides consist of more than one transcriptional regulatory polynucleotide.

In some embodiments, the polynucleotide segments are single-stranded. In some embodiments, the polynucleotide segments are double-stranded. In some embodiments, the double-stranded segments have at least one overhanging single-stranded end. In some embodiments, the overhanging single-stranded end comprises fewer than 10 base pairs.

In some embodiments, the assembling step does not comprises a polymerase.

The invention also provides a reassembled polynucleotide assembled by the above-described methods.

### Definitions

The phrases “nucleic acid sequence” or “polynucleotide” refer to a single or double-stranded polymer of deoxyribonucleotide or ribonucleotide bases read from the 5′ to the 3′ end. It includes chromosomal DNA, self-replicating plasmids and infectious polymers of DNA or RNA.

“Combinatorially reassembled” or “reassembled” polynucleotides refer to nucleic acid molecules that are the product of the combination of DNA segments.

A “transcriptional regulatory polynucleotide” is any polynucleotide that acts to modulate transcription of a gene. Examples of transcriptional regulatory elements include promoters, enhancers and cis-acting sequences that act alone, or in combination, to regulate transcription.

“Progenitor” refers to polynucleotides that are employed in the present invention as a source of nucleic acid segments.

The term “promoter” is used herein to refer to an array of nucleic acid control sequences that direct transcription of an operably linked nucleic acid. Promoters include nucleic acid sequences near the start site of transcription, such as, in the case of a polymerase II type promoter, a TATA element. Promoters also include cis-acting polynucleotide sequences that can be bound by transcription factors. A promoter also optionally includes distal “enhancer” or repressor elements, which can be located as much as several thousand base pairs from the start site of transcription. Enhancer or repressor elements regulate transcription in an analogous manner to cis-acting elements near the start site of transcription, with the exception that enhancer elements can act from a distance from the start site of transcription.

A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, plant organs (e.g., leaves, stems, flowers, roots, etc.), seeds and plant cells and progeny of same. The class of plants which can be used in the method of the invention is generally as broad as the class of flowering plants amenable to transformation techniques, including angiosperms (monocotyledonous and dicotyledonous plants), as well as gymnosperms. It includes plants of a variety of ploidy levels, including polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is different from any naturally occurring allelic variants.

An “expression cassette” refers to a polynucleotide with a series of nucleic acid elements that permit transcription of a particular nucleic acid, e.g., in a cell. Typically, the expression cassette includes a nucleic acid to be transcribed operably linked to a promoter.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA). The term “absolute percent identity” refers to a percentage of sequence identity determined by scoring identical amino acids as 1 and any substitution as zero, regardless of the similarity of mismatched amino acids. In a typical sequence alignment, e.g., a BLAST alignment, the “absolute percent identity” of two sequences is presented as a percentage of amino acid “identities.” As used herein, where a sequence is defined as being “at least X % identical” to a reference sequence, e.g., “a polypeptide at least 90% identical to SEQ ID NO:2,” it is to be understood that “X % identical” refers to absolute percent identity, unless otherwise indicated. In cases where an optimal alignment of two sequences requires the insertion of a gap in one or both of the sequences, an amino acid residue in one sequence that aligns with a gap in the other sequence is counted as a mismatch for purposes of determining percent identity. Gaps can be internal or external, i.e., a truncation.

The term “substantial identity” of polynucleotide sequences means that a polynucleotide comprises a sequence that has at least 25% sequence identity. Alternatively, percent identity can be any integer from at least 25% to 100% (e.g., at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%). Some embodiments include at least: 25%, 30%, 35%, 40%, 45%, 50%, 55%, 60%, 65%, 70%, 75%, 80%, 85%, 90%, 95%, or 99% compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001,

Another indication that two nucleic acid sequences are substantially identical is that the two molecules or their complements hybridize to each other under stringent conditions, as described below. The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

In the present invention, genomic DNA or cDNA comprising nucleic acids of the invention can be identified in standard Southern blots under stringent conditions using the nucleic acid sequences disclosed here. Moreover, in certain embodiments, two or more polynucleotides (e.g., two transcriptional regulatory polynucleotides) do not hybridize under stringent conditions. For the purposes of this disclosure, suitable stringent conditions for such hybridizations are those which include a hybridization in a buffer of 40% formamide, 1 M NaCl, 1% SDS at 37° C., and at least one wash in 0.2×SSC at a temperature of at least about 50° C., usually about 55° C. to about 60° C. or 60° C., for 20 minutes, or equivalent conditions. A positive hybridization is at least twice background. Those of ordinary skill will readily recognize that alternative hybridization and wash conditions can be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., a northern or Southern blot.

### DETAILED DESCRIPTION

The present invention provides methods useful for obtaining a polynucleotide with transcriptional activity. In particular, the invention demonstrates for the first time, the surprising finding that, without regard to specific known or unknown cis-acting sequences, random polynucleotide segments can be ligated in a random fashion to produce a reassembled polynucleotide with a different transcriptional regulatory activity than the progenitor polynucleotide(s) from which the segments were derived.

By using random polynucleotide segments from transcriptional regulatory progenitor polynucleotides, novel cis-acting sequences can be formed by combining parts of cis-acting sequences that result from the random selection process. For example, at some frequency, due to the random nature of how the segments are constructed, a part of a cis-acting sequence from a progenitor polynucleotide can be combined with parts from other cis-acting sequences, or random sequences, to form a novel cis-acting sequence. Such novel cis-acting sequences would not be formed by combining whole cis-acting sequences only.

Indeed, by obtaining the segments randomly, a much larger number of different segments can be combined than can possibly be formed by combining only known cis-acting elements. In turn, the large number of segments allows for the construction of libraries of a significant number of reassembled polynucleotides, each potentially having novel transcriptional regulatory activity. Efficient methods for identifying polynucleotides can subsequently be designed to screen the numerous combinations for a particular transcriptional regulatory activity of interest.

Generally, both positive and negative cis-acting regulatory regions co-exist within a promoter region. In order to enhance promoter activity, one needs to increase the number of positive elements and decrease the number of negative elements. Alternatively, inserting an element that has higher affinity for positively-acting transcription factors can be effective to increase promoter activity. In some embodiments, these studies are effective for designing tissue-specific promoters that already tend to be lower in activity than high-activity constitutive promoters. See, e.g., Nettlebeck, et al., Trends Genet. 16(4):174-81 (2000). As the identity and location of cis-acting regulatory elements within a promoter are generally not known, the recombination of random DNA segments within a promoter combined with a defined activity screen offers a solution for creating promoters with desired properties. The typical length of enhancer region DNA protected by a particular transcription factor is 20-30 base pairs in length. The core recognition sequences within these enhancer elements may only be 5 or fewer base pairs in length. Thus, reconstruction of promoters by a random fragmentation, mutagenesis, and assembly approach is useful. One may find, for example, that a promoter of enhanced function contains not a few or no silencing elements and more enhancing elements. Moreover, novel enhancer elements are also synthesized by this method. Also, the simultaneous introduction of mutations in the parent molecules prior to recombination increases the diversity of possible enhancer element structures. In contrast, the combinatorial assembly of known enhancer elements would not provide for discovery of hybrid enhancer elements.

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., (1989) or Ausubel et al., eds., Current Protocols, a joint venture between Greene Publishing Associates, Inc. and John Wiley & Sons, Inc., (1995 Supplement) (Ausubel).

I. Polynucleotide Segments of the Invention

As described below, segments are typically derived from progenitor polynucleotides with transcriptional regulatory activity. A number of methods for obtaining random polynucleotide segments of the invention are known to those of skill in the art. Segments are obtained without regard to specific sequences in the progenitor polynucleotide. Indeed, in one aspect of the present invention, cis-acting sequences in a progenitor polynucleotide are recombined to create a cis-acting sequence that is not found in the progenitor polynucleotide. Random sequences can be obtained, for example, by randomly cleaving the progenitor polynucleotides or by randomly amplifying parts of the progenitor sequences.

The polynucleotide segments can be of various lengths depending on the size of the promoter or enhancer to be recombined or reassembled. In some embodiments, the sequences are less than about 20,000 bp long. In some embodiments, the sequences are from about 5 bp to about 5,000 bp long. In some embodiments, the segments are between about 5 to about 20 base pairs or about 10 bp to 1,000 bp. In some embodiments, the segments are about 20 bp to about 500 bp. In some embodiments, the segments are greater than, e.g., about 20, 50, 100, 200, 500, 1000 or more base pairs. In some embodiments, the segments have fewer than about 10000, 5000, 1000, 500, 200, 100, or 50 base pairs.

Any number of segments can be assembled at one time. In some embodiments, the number of segments range from about 3 to about 10,000 segments. In some embodiments, the number of segments range from about 5 to about 500 segments. In some embodiments, the number of segments range from about 10 to about 100 segments. In some embodiments, the number of segments is more than about 3, 5, 10, 20 or more fragments. In some embodiments, the number of segments is fewer than about 10000, 1000, 500, 100 or 50 fragments.

The polynucleotide segments can be single-stranded or double-stranded. Double-stranded segments can have one or two ends that comprise single-stranded overhangs. Single-stranded overhangs can be, for example, 1, 2, 3, 4, 5, 6, 7, 8, 9, 10, 12, 15, 20 or more base pairs long.

The resulting reassembled polynucleotide can be of various lengths. Preferably the reassembled sequences are from about 50 bp to about 10 kb.

Random Cleaving

Any means of cleaving DNA molecules can be used to produce segments of the invention. For example, a well-known method of randomly cleaving DNA comprises shearing DNA using mechanical force. See, e.g., Sambrook et al., Molecular Cloning—A Laboratory Manual, Cold Spring Harbor Laboratory, Cold Spring Harbor, N.Y., (1982 and 1989). Alternatively, sequence specific or non-specific DNA cleaving enzymes can be used to cleave a progenitor polynucleotide. Examples of sequence-specific enzymes useful in the methods of the invention comprise restriction enzymes that bind and cleave at or near a specific polynucleotide sequence. The length of the recognition sequence determines the average length of desired segments. For example, restriction enzymes that recognize four base pair sequences will cleave a particular polynucleotide, on average, more frequently (and therefore produce a shorter average segment) than a restriction enzyme that recognizes a five or six base pair recognition sequence. Of course, different progenitor polynucleotides will be cleaved into different segments of different lengths. Therefore, in some embodiments more than one restriction enzyme is used either individually, or in combination, to create segments of the desired length corresponding to a region of the polynucleotide. One possible restriction enzyme is CviTI, which recognizes a particular three base pair sequence.

In some embodiments, restriction enzymes that produce “sticky ends,” i.e., complementary single-stranded ends, are used. Enzymes capable of filling in single stranded gaps in sequences (“fill in enzymes”) are also employed in some embodiments. Such enzymes include klenow fragment and T4 polymerase.

In some embodiments, non-specific DNA cleaving enzymes are employed to create segments of the invention. For example, DNaseI, which cleaves DNA without regard to a particular polynucleotide sequence, can be used in the methods of the invention. Those of skill in the art will recognize that the time of exposure of an active non-specific DNA cleaving enzyme to progenitor polynucleotides will determine the resulting average segment length. Such reactions are typically stopped after a desired time by, e.g., denaturing the enzyme by raising the temperature of the reaction.

Non-specific DNA cleaving enzymes can also be used in conjunction with enzymes such as klenow fragment and T4 polymerase. Other enzymes useful for generating diverse segments include, e.g., uracil-N-glycosylase or nickase, with or without fill in enzymes.

Random Amplification

Any method of amplification can be used to produce segments for reassembling. A method for amplification of DNA segments combines the use of synthetic oligonucleotide primers, including random priming, as discussed below, and amplification of a DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences directly from genomic libraries. Restriction endonuclease sites can be incorporated into the primers to improve the efficiency of the ligation step (see below).

Generally, segments are generated by using random primers, typically no longer than ten nucleotides long, that are subsequently used to amplify segments. Preferably, primers are between about six nucleotides to about ten nucleotides in length.

In some embodiments, additional diversity is introduced into the segment sequences by amplifying the segments using an error-prone amplification technique. Examples of mutagenic amplification techniques are discussed in, e.g., Shafikhani, S., et al. (1997) BioTechniques 23: 304-306 and Stemmer, W. P. (1994) Proc. Natl. Acad. Sci. USA 91:10747-10751.

Progenitor Polynucleotides

Any DNA polynucleotide sequence (i.e., progenitor polynucleotides) can be used to derive the segments for reassembling. Indeed, in some embodiments, more than one polynucleotide sequence can be used. In some embodiments, the polynucleotides are promoter or enhancer (i.e., transcriptional regulatory) polynucleotide sequences. In some embodiments, the polynucleotides are transcriptional regulatory sequences known to have a particular activity. For instance, a specific promoter sequence may be identified for its ability to initiate transcription at a particular level (high or low expression) or can be cell- or tissue-specific or inducible. In some embodiments, the polynucleotides are selected from gene homologs from different species. In some embodiments, the different promoters with the same promoter specificity are selected. Alternatively, promoters with different promoter specificity are selected.

Methods for identification of promoters from polynucleotides comprising gene sequences are well known to those of skill in the art. Sequence motifs associated with promoters, such as the TATA box in eukaryotes, or the TATA box and −35 consensus sequence (TGTTGACA) in prokaryotes, can be used to identify the general region of a promoter. Moreover, various techniques for promoter analysis such as deletion analysis can be used to determine the minimal region required for transcriptional activity. Linker-scan mutagenesis can also be used to identify regions of a polynucleotide that are required for transcriptional activity. Typically, this analysis is performed by ligating the candidate promoter sequence to a reporter gene construct, as discussed below.

Examples of particular progenitor promoter polynucleotides include promoters from yeast, fungi, bacteria, viruses, plants, or animals, including mammals. Constitutive, tissue- or cell-specific or inducible promoters, among others, can be used as a progenitor polynucleotide.

a. Constitutive Promoters

In some embodiments, a promoter segment is employed which directs expression of the genes in all tissues of an organism. Such promoters are referred to herein as “constitutive” promoters and are active under most environmental conditions and states of development or cell differentiation. Examples of constitutive plant promoters include the cauliflower mosaic virus (CaMV) 35S transcription initiation region, as well as other Pararetrovirus-like 35S promoters, the 1′- or 2′-promoter derived from T-DNA of Agrobacterium tumafaciens, the ubiquitin promoter, and other transcription initiation regions from various plant genes known to those of skill. Such genes include for example, Act2 or Act8 from Arabidopsis (An et al., Plant J 10:107-121 (1996)), and Cat3 from Arabidopsis (GenBank No. U43147, Zhong et al., Mol. Gen. Genet. 251:196-203 (1996)). Additional constitutive promoters include the A1 EF-1A promoter (Curie, et al., Mol. Gen. Genet. 238:428-436 (1993)), the atpkl promoter (Zhang et al., J. Biol. Chem. 269:17586-17592 (1994)), the UBQ3 promoter (Norris et al., Plant Mol. Biol. 21:895-906 (1993)), the NelF4A10 promoter (Mandel et al., Plant Mol. Biol. 29:995-1004 (1995)), the TUA2 promoter (Carpenter et al., Plant Mol. Biol. 21:937-942 (1993)), the A-p40 promoter (Scheer et al., Plant Mol. Biol. 35:905-913 (1997)), the HMG-I/Y promoter (Gupta, et al., Plant Mol. Biol. 36:897-907 (1998)), the AAP19-1 promoter (Maldonado-Mendoza, et al., Plant Mol. Biol. 35:865-872 (1997)) and the apt promoter (Maffat, et al., Gene 143:211-216 (1994)).

Examples of mammalian promoters include CMV promoter, SV40 early promoter, SV40 later promoter, metallothionein promoter, murine mammary tumor virus promoter, Rous sarcoma virus promoter, polyhedrin promoter, or other promoters shown effective for expression in animal cells.

b. Cell- and Tissue-Specific Promoters

Alternatively, one or more progenitor polynucleotides can direct expression in a specific tissue or may be otherwise under more precise environmental or developmental control. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

Examples of plant promoters under developmental control include promoters that initiate transcription only (or primarily only) in certain tissues, such as fruit, seeds, or flowers. For example, suitable seed specific promoters include those derived from the following genes: MAC1 from maize (Sheridan et al. Genetics 142:1009-1020 (1996), Cat3 from maize (GenBank No. L05934, Abler et al. Plant Mol. Biol. 22:10131-1038 (1993), the gene encoding oleosin 18 kD from maize (GenBank No. J05212, Lee et al. Plant Mol. Biol. 26:1981-1987 (1994)), vivparous-1 from Arabidopsis (Genbank No. U93215), the gene encoding oleosin from Arabidopsis (Genbank No. Z17657), Atmyc1 from Arabidopsis (Urao et al. Plant Mol. Biol. 32:571-576 (1996), the 2s seed storage protein gene family from Arabidopsis (Conceicao et al. Plant 5:493-505 (1994)) the gene encoding oleosin 20 kD from Brassica napus (GenBank No. M63985), napA from Brassica napus (GenBank No. J02798, Josefsson et al. JBL 26:12196-1301 (1987), the napin gene family from Brassica napus (Sjodahl et al. Planta 197:264-271 (1995), the gene encoding the 2S storage protein from Brassica napus (Dasgupta et al. Gene 133:301-302 (1993)), the genes encoding oleosin A (Genbank No. U09118); oleosin B (Genbank No. U09119) from soybean and the gene encoding low molecular weight sulphur rich protein from soybean (Choi et al. Mol Gen, Genet. 246:266-268 (1995)); ACT1 from Arabidopsis (Huang et al. Plant Mol. Biol. 33:125-139 (1996)); the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), and Gpc2 from maize (GenBank No. U45855, Manjunath et al., Plant Mol. Biol. 33:97-112 (1997)).

Other plant examples include promoters from the actin, tubulin and EF1a gene families (Manevski, et al.: FEBS Lett 483(1):43-46 (2000)), each of which contain members that are active only in actively-growing cells. EF1a is particularly active in meristematic cells. Other plant tissue-specific promoters include the SSU promoter (Gittins, et al. Planta 210(2):232-40 (2000)), which is specific for green tissues and is light regulated. The Napin (Stalberg, et al. Planta 199(4):515-9 (1996)), 7S albumin and 2S albumin promoters are additional seed-specific promoters. The E8 promoter (Good, et al.: Plant Mol Biol (3):781-90 (1994)) is tomato fruit-specific.

Examples of tissue-specific promoters for animal cells include the promoter for creatine kinase, which has been used to direct the expression of dystrophin cDNA expression in muscle and cardiac tissue (Cox, et al. Nature 364:725-729 (1993)) and immunoglobulin heavy or light chain promoters for the expression of suicide genes in B cells (Maxwell, et al. Cancer Res. 51:4299-4304 (1991)). An endothelial cell-specific regulatory region has also been characterized (Jahroudi, et al. Mol Cell. Biol. 14:999-1008 (1994)). Amphotrophic retroviral vectors have been constructed carrying a herpes simplex virus thymidine kinase gene under the control of either the albumin or alpha-fetoprotein promoters (Huber, et al. Proc. Natl. Acad. Sci. U.S.A. 88:8039-8043 (1991)) to target cells of liver lineage and hepatoma cells, respectively.

The human smooth muscle-specific alpha-actin promoter is discussed in Reddy, et al., J. Cell Biology 265:1683-1687 (1990) which discloses the isolation and nucleotide sequence of this promoter, while Nakano, et al., Gene 99:285-289 (1991) discloses transcriptional regulatory elements in the 5′ upstream and the first intron regions of the human smooth muscle (aortic type) alpha-actin gene. Petropoulos, et al., J. Virol. 66:3391-3397 (1992)) disclose a comparison of expression of bacterial chloramphenicol transferase (CAT) operatively linked to either the chicken skeletal muscle alpha actin promoter or the cytoplasmic beta-actin promoter.

Exemplary tissue-specific expression elements for the liver include but are not limited to HMG-COA reductase promoter (Luskey, Mol. Cell. Biol. 7(5):1881-1893 (1987)); sterol regulatory element 1 (SRE-1; Smith et al. J. Biol. Chem. 265(4):2306-2310 (1990); phosphoenol pyruvate carboxy kinase (PEPCK) promoter (Eisenberger et al. Mol. Cell Biol. 12(3):1396-1403 (1992)); human C-reactive protein (CRP) promoter (Li et al. J. Biol. Chem. 265(7):4136-4142 (1990)); human glucokinase promoter (Tanizawa et al. Mol. Endocrinology 6(7):1070-81 (1992); cholesterol 7-alpha hydroylase (CYP-7) promoter (Lee et al. J. Biol. Chem. 269(20): 14681-9 (1994)); beta-galactosidase alpha-2,6 sialyltransferase promoter (Svensson et al. J. Biol. Chem. 265(34):20863-8 (1990); insulin-like growth factor binding protein (IGFBP-1) promoter (Babajko et al. Biochem Biophys. Res. Comm. 196 (1):480-6 (1993)); aldolase B promoter (Bingle et al. Biochem J 294(Pt2):473-9 (1993)); human transferrin promoter (Mendelzon et al. Nucl. Acids Res. 18(19):5717-21 (1990); collagen type I promoter (Houglum et al. J. Clin. Invest. 94(2):808-14 (1994)).

Exemplary tissue-specific expression elements for the prostate include but are not limited to the prostatic acid phosphatase (PAP) promoter (Banas et al. Biochim. Biophys. Acta. 1217(2):188-94 (1994); prostatic secretory protein of 94 (PSP 94) promoter (Nolet et al. Biochim. Biophys. ACTA 1089(2):247-9 (1991)); prostate specific antigen complex promoter (Kasper et al. J. Steroid Biochem. Mol. Biol. 47 (1-6):127-35 (1993)); human glandular kallikrein gene promoter (hgt-1) (Lilja et al. World J. Urology 11(4):188-91 (1993).

Exemplary tissue-specific expression elements for gastric tissue include those discussed in Tamura et al. FEBS Letters 298: (2-3):137-41 (1992).

Exemplary tissue-specific expression elements for the pancreas include but are not limited to pancreatitis associated protein promoter (PAP) (Dusetti et al. J. Biol. Chem. 268(19):14470-5 (1993)); elastase 1 transcriptional enhancer (Kruse et al. Genes and Development 7(5):774-86 (1993)); pancreas specific amylase and elastase enhancer promoter (Wu et al. Mol. Cell. Biol. 11(9):4423-30 (1991); Keller et al. Genes & Dev. 4(8):1316-21 (1990)); pancreatic cholesterol esterase gene promoter (Fontaine et al. Biochemistry 30(28):7008-14 (1991)).

Exemplary tissue-specific expression elements for the endometrium include but are not limited to the uteroglobin promoter (Helftenbein et al. Annal. NY Acad. Sci. 622:69-79 (1991)).

Exemplary tissue-specific expression elements for adrenal cells include but are not limited to cholesterol side-chain cleavage (SCC) promoter (Rice et al. J. Biol. Chem. 265:11713-20 (1990).

Exemplary tissue-specific expression elements for the general nervous system include but are not limited to gamma-gamma enolase (neuron-specific enolase, NSE) promoter (Forss-Petter et al. Neuron 5(2):187-97 (1990)).

Exemplary tissue-specific expression elements for the brain include but are not limited to the neurofilament heavy chain (NF—H) promoter (Schwartz et al. J. Biol. Chem. 269(18):13444-50 (1994)).

Exemplary tissue-specific expression elements for lymphocytes include but are not limited to the human CGL-1/granzyme B promoter (Hanson et al. J. Biol. Chem. 266 (36):24433-8 (1991)); the terminal deoxy transferase (TdT), lambda 5, VpreB, and Ick (lymphocyte specific tyrosine protein kinase p56lck) promoter (Lo et al. Mol. Cell. Biol. 11(10):5229-43 (1991)); the humans CD2 promoter and its 3′ transcriptional enhancer (Lake et al. EMBO J. 9(10):3129-36 (1990)), and the human NK and T cell specific activation (NKG5) promoter (Houchins et al. Immunogenetics 37(2):102-7 (1993)).

Exemplary tissue-specific expression elements for the colon include but are not limited to pp60c-src tyrosine kinase promoter (Talamonti et al. J. Clin. Invest 91(1):53-60 (1993)); organ-specific neoantigens (OSNs), mw 40 kDa (p40) promoter (Ilantzis et al. Microbiol. Immunol. 37(2):119-28 (1993)); colon specific antigen-P promoter (Sharkey et al. Cancer 73(3 supp.) 864-77 (1994)).

Exemplary tissue-specific expression elements for breast cells include but are not limited to the human alpha-lactalbumin promoter (Thean et al. British J Cancer. 61(5):773-5 (1990))

Other tissue-specific promoters include the phosphoeholpyruvate carboxykinase (PEPCK) promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, alpha or beta globin promoter, T-cell receptor promoter, the osteocalcin promoter the IL-2 promoter, IL-2 receptor promoter, whey (wap) promoter, and the MHC Class II promoter.

Fungal promoters that are regulated by external or internal factors include the PGAL1 promoter (Farfan, et al. Appl Environ Microbiol 65(1): 110-6 (1999)) and others that are well known in the art.

c. Inducible Promoters

Examples of environmental conditions that may effect transcription by inducible promoters include anaerobic conditions, elevated temperature, a particular chemical compound or the presence of light. Such promoters are referred to here as t “inducible” promoters. For instance, inducible promoters include the glucocorticoid-inducible promoter described in McNellis et al., Plant J. 14(2):247-57 (1998). U.S. Pat. No. 5,877,018 describes metal responsive and glucocorticoid-responsive promoter elements. Other inducible promoters include the pathogenesis-related gene promoters including the PR-1 promoter (Uknes, et al. Plant Cell 5(2):159-69 (1993); Meier et al., Plant Cell 3(3):309-15 (1991)), which is induced by salicylic acid in plants.

Hormones that have been used to regulate gene expression include, for example, estrogen, tomoxifen, toremifen and ecdysone (Ranikumar and Adler Endocrinology 136: 536-542 (1995)). See, also, Gossen and Bujard Proc. Natl. Acad. Sci. USA 89: 5547 (1992); Gossen et al. Science 268:1766 (1995). In tetracycline-inducible systems, tetracycline or doxycycline modulates the binding of a repressor to the promoter, thereby modulating expression from the promoter. An additional example includes the ecdysone responsive element (No et al., Proc. Nat'l. Acad. Sci. USA 93:3346 (1997)). Other examples of inducible promoters include the glutathione-S-transferase II promoter which is specifically induced upon treatment with chemical safeners such as N,N-diallyl-2,2-dichloroacetamide (PCT Application Nos. WO 90/08826 and WO 93/01294) and the alcA promoter from Aspergillus, which in the presence of the alcR gene product is induced with cyclohexanone (Lockington, et al., Gene 33:137-149 (1985); Felenbok, et al. Gene 73:385-396 (1988); Gwynne, et al. Gene 51:205-216 (1987)) as well as ethanol. Other examples include promoters induced in response to infection or disease.

Isolation of the Polynucleotides of the Invention

The isolation of nucleic acids of the invention may be accomplished by a number of techniques. For instance, oligonucleotide probes based on known sequences can be used to identify the desired gene in genomic DNA library. To construct genomic libraries, large segments of genomic DNA are generated by random fragmentation, e.g. using restriction endonucleases, and are ligated with vector DNA to form concatemers that can be packaged into the appropriate vector.

The genomic library can then be screened using a probe based upon the sequence of a cloned gene of the invention. Probes may be used to hybridize with genomic DNA or cDNA sequences to isolate homologous genes in the same or different species. Isolated cDNA sequences can be used as probes to identify genomic clones and therefore, associated transcriptional regulatory elements.

Alternatively, the nucleic acids of interest can be amplified from nucleic acid samples using amplification techniques. For instance, polymerase chain reaction (PCR) technology can be used to amplify the sequences of the polynucleotides of the invention directly from genomic DNA, or from genomic libraries. PCR and other in vitro amplification methods may also be useful, for example, to clone promoter or enhancer sequences, as well as to clone nucleic acid sequences that code for proteins to be expressed, to make nucleic acids to use as probes for detecting the presence of the desired mRNA in samples, for nucleic acid sequencing, or for other purposes. For a general overview of PCR see PCR Protocols: A Guide to Methods and Applications. (Innis, M, Gelfand, D., Sninsky, J. and White, T., eds.), Academic Press, San Diego (1990).

Appropriate primers and probes for identifying sequences of the invention from an organism of interest are generated from comparisons with desired sequences or other related sequences. Using these techniques, one of skill can identify conserved regions in the nucleic acids of the invention to prepare the appropriate primer and probe sequences. Primers that specifically hybridize to conserved regions in genes of the invention can be used to amplify sequences from widely divergent species.

Exemplary amplification conditions include, e.g., the following reaction components: 10 mM Tris-HCl, pH 8.3, 50 mM potassium chloride, 1.5 mM magnesium chloride, 0.001% gelatin, 200 μM dATP, 200 μM dCTP, 200 μM dGTP, 200 μM dTTP, 0.4 μM primers, and 100 units per ml Taq polymerase. Program: 96 C for 3 min., 30 cycles of 96 C for 45 sec., 50 C for 60 sec., 72 for 60 sec, followed by 72 C for 5 min. Those of skill in the art will recognize that other reaction conditions can be used to obtain similar results.

Standard nucleic acid hybridization techniques using the conditions disclosed above can then be used to identify genomic clones.

Oligonucleotides

In some embodiments of the invention, single or double stranded oligonucleotide primers can be added to the assembly reaction to provide additional diversity in the resulting reassembled polynucleotides. Preferably, the oligonucleotides comprise known protein binding sequences or regions of DNA where deletion or mutational analysis indicates a functional element exists. Selection of such sequences is based on the type of transcriptional activity to be identified. For example, oligonucleotides comprising inducible cis-acting elements can be introduced if inducible promoters are desired. See, e.g., U.S. Pat. No. 5,877,018. In some embodiments, oligonucleotides have fewer than 100, 50, 40, 30, 20 or 10 nucleotides.

II. Assembling Segments of the Invention

In some embodiments, reassembled polynucleotides of the invention are constructed by combining segments in a random manner. For example, segments for the construction of a reassembled polynucleotide can be ligated in a reaction with the appropriate buffers and a DNA ligase (e.g., T4 ligase, etc.) and then cloned into a plasmid vector.

Efficient ligation of the segments depends on the nature of the ends of the segments. Compatible “sticky” ends or blunt ends of segments can be efficiently ligated. In cases where some or all of the ends are not compatible or blunt, the segments can be treated (e.g., with Klenow fragment and or T4 DNA polymerase) to insure that all segments have a blunt end. Alternatively, specific adaptor oligonucleotide sequences can be added to improve the efficiency of the ligation reaction.

In some embodiments, polynucleotide fragments are recombined by linking overlapping single stranded segments and then contacting the resulting linked segments with a polymerase. For example, the polymerase chain reaction can be used to amplify and thereby recombine the overlapping segments. See, e.g., U.S. Pat. No. 6,150,111.

In other aspects, recombination is independent of natural restriction sites or in vitro ligation (Ma et al., Gene 58:201-216 (1989); Oldenburg et al., Nucleic Acids Research 25:451-452 (1997)). In some of these methods, an in vivo method for plasmid construction takes advantage of the double-stranded break repair pathway in a cell such as a yeast cell to achieve precision joining of DNA fragments. This method involves synthesis of linkers (, e.g., 60-140 base pairs) from short oligonucleotides and requires assembly by enzymatic methods into the linkers needed (Raymond et al., BioTechniques 26(1): 134-141 (1999)).

In some aspects, short random or non-random oligonucleotide sequences are recombined with polynucleotide segments derived from transcriptional regulatory polynucleotides. In some embodiments, the oligonucleotides comprise polynucleotide sequences that are recognized by transcription factors or other transcriptional regulatory proteins.

In some embodiments, modifications are introduced into the polynucleotide segments or the recombined polynucleotides. For example, the polynucleotides can be submitted to one or more rounds of error-prone PCR (e.g., Leung, D. W. et al., Technique 1:11-15 (1989); Caldwell, R. C. and Joyce, G. F. PCR Methods and Applications 2:28-33 (1992); Gramm, H. et al., Proc. Natl. Acad. Sci. USA 89:3576-3580 ( 1992)), thereby introducing variation into the polynucleotides. Alternatively, cassette mutagenesis (e.g., Stemmer, W. P. C. et al., Biotechniques 14:256-265 (1992); Arkin, A. and Youvan, D. C. Proc. Natl. Acad. Sci. USA 89:7811-7815 (1992); Oliphant, A. R. et al., Gene 44:177-183 (1986); Hermes, J. D. et al., Proc. Natl. Acad. Sci. USA 87:696-700 (1990)), in which the specific region to be optimized is replaced with a synthetically mutagenized oligonucleotide, can be used. Mutator strains of host cells can also be employed to add to mutational frequency (Greener and Callahan, Strategies in Mol. Biol. 7: 32 (1995)).

Once the polynucleotides are assembled, the polynucleotides can be cloned into a vector comprising a minimal promoter operably linked to a reporter gene. In this manner, libraries of reassembled promoter candidates can be created and subsequently stored for future screening.

III. Selecting Reassembled Polynucleotides of the Invention

The methods of the invention can be used to improve or alter the properties of promoters/enhancers from genes from any type of organism. The way that a particular reassembled promoter is selected is determined by the type of promoter desired. A general method for selecting promoters comprises introducing the reassembled promoter into a basal or minimal promoter construct that is operably linked to a reporter gene. By testing constructs of the invention for reporter gene activity under desired conditions and cell types, a reassembled polynucleotide that confers an improved or desired transcriptional activity can be determined. Selection of cells or organisms to test the contructs of the invention is determined by the desired promoter activity.

In some embodiments, particularly where a high-expression promoter is desired, an organism (e.g., a plant), cell line, or individual cells/protoplasts are transformed with candidate reassembled promoters operably linked to a reporter gene (e.g., encoding green fluorescent protein (GFP)) and transformants are analyzed for reporter activity (e.g., fluorescence) in tissues where promoter activity is desired. In other embodiments where tissue-specific expression is desired in a seed of a plant, plant lines with clear seed coats are selected (e.g., tt mutants in Arabidopsis) and candidate promoters operably linked to a visual marker (e.g., GFP, lycopene, β-carotene, etc.) are transformed into such plants. Seed harvested from the primary transformants with seed-specific promoters are recognized by a change of color in the seed.

Similarly, fruit-specific promoters can be identified in tomato fruit by operably linking a reporter gene to promoter candidates and transforming tomato. A useful variety of tomato for this procedure is a “microtom” variety.

Minimal Promoters

A minimal or basal promoter will typically comprise a TATA box and transcriptional start sequence, but will not contain additional stimulatory and repressive elements. An exemplary plant minimal promoter is positions −50 to +8 of the 35S CaMV promoter. Exemplary animal minimal promoters include the SV40 early minimal promoter and the CMV promoter from positions −53 to +75 (Gossen, et al. Proc. Natl. Acad. Sci. USA 89:5547 (1992)). A fungal minimal promoter can be obtained from the TATA box region of the Saccharomycetes cerevisiae iso-1-cytochrome c (cyc1) promoter, as well as the GAL1 promoter. A bacterial minimal promoter includes the lacZ minimal promoter.

In one embodiment of the present invention, polynucleotide segments derived from one or more progenitor transcriptional regulatory polynucleotides are assembled and operably linked to a specific minimal promoter. In some embodiments, the polynucleotide segments are derived from transcriptional regulatory polynucleotides that exclude minimal promoter sequences.

Reporter Genes

Reporter genes are generally useful for analyzing the transcriptional activity of a candidate promoter. Reporter genes are operably linked to a candidate promoter and then expressed. The protein encoded by the reporter gene typically produces a detectable product which can be compared visually or analytically (e.g., by ELISA). Alternatively, the quantity of the product can be determined by measuring light absorbance, fluorescence, or luminescence at a specific wavelength of a sample. Examples of reporter systems include luciferase (Cohn et al., Proc. Natl. Acad. Sci. USA 80:102-123 (1983); U.S. Pat. No. 5,196,524), β-galactosidase (Jefferson, et al., Proc. Natl. Acad. Sci. USA 83:8447-8451 (1986)), β-glucuronidase (GUS) (GUS PROTOCOLS: USING THE GUS GENE AS A REPORTER OF GENE EXPRESSION (ed. Gallagher) Academic Press, New York 1992) and green fluorescent protein (see, e.g., U.S. Pat. Nos. 5,491,084 and 5,958,713).

### EXAMPLES

The following examples are offered to illustrate, but not to limit the claimed invention.

### Example 1

Single promoter “assembly” of the Aspergillus alcohol dehydrogenase 1 (AlcA) promoter is carried out to identify variants with higher expression levels in response to the AlcR trans-activator protein.

A 325-base pair region of the AlcA promoter is amplified by the polymerase chain reaction from Aspergillus nidulans genomic DNA. The cloned PCR product is then cut into segments using a series of restriction enzymes that leave blunt ends. The segments are randomly assembled using T4 DNA ligase and cloned into a yeast expression vector containing a minimal TATA box region and a reporter gene.

The vector library of reassembled variants is transformed into a yeast strain that expresses the AlcR protein from an integrated DNA element. Colonies are screened for expression of the reporter gene. Colonies with greater reporter expression than the progenitor AlcA promoter-reporter control strain are further characterized to quantify the level of promoter improvement.

### Example 2

Multiple promoter “assembly” of the Aspergillus alcohol dehydrogenase 1 (AlcA), aldehyde dehydrogenase 1 (aldA), and Alc regulatory protein (AlcR) promoters is carried out to identify variants with higher expression levels in response to the AlcR trans-activator protein.

Approximately 350-base pair regions of the AlcA, AldA, and AlcR promoters are amplified by the polymerase chain reaction from Aspergillus genomic DNA. The cloned PCR products are cleaved into random segments using CviTI* restriction endonuclease under relaxed conditions (Megabase Research Products). The segments are randomly assembled using T4 DNA ligase and cloned into a yeast expression vector containing a minimal TATA box region and a reporter gene.

The vector library of reassembled variants is then transformed into a yeast strain that expresses the AlcR protein from an integrated DNA element. Colonies are screened for expression of the reporter gene. Colonies with greater reporter expression than the progenitor AlcA promoter-reporter control strain are further characterized to quantify the level of promoter improvement.

### Example 3

Single promoter “assembly” with oligonucleotide spiking of the Aspergillus alcohol dehydrogenase 1 (AlcA) promoter is carried out to identify variants with higher expression levels in response to the AlcR trans-activator protein.

A 325-base pair region of the AlcA promoter is amplified by the polymerase chain reaction from Aspergillus genomic DNA. The cloned PCR product is cut into segments using a series of restriction enzymes that leave blunt ends. A short double-stranded oligonucleotide is designed that corresponds in sequence to a known DNA binding site for the AlcR regulatory protein. The segments and oligonucleotide are randomly assembled using T4 DNA ligase and cloned into a yeast expression vector containing a minimal TATA box region and a reporter gene.

The vector library of reassembled variants is transformed into a yeast strain that expresses the AlcR protein from an integrated DNA element. Colonies are screened for expression of the reporter gene. Colonies with greater reporter expression than the progenitor AlcA promoter-reporter control strain are further characterized to quantify the level of promoter improvement.

### Example 4

Single promoter “assembly” of mutated promoter elements from the Aspergillus alcohol dehydrogenase 1 (AlcA) gene is carried out to identify variants with higher expression levels in response to the AlcR trans-activator protein.

A 325-base pair region of the AlcA promoter is amplified by the polymerase chain reaction from Aspergillus genomic DNA. Additional diversity is introduced into the sequence by using mutagenic amplification techniques such as error-prone PCR with an unbalanced nucleotide ratio. The cloned PCR products are cut into segments using a series of restriction enzymes that leave blunt ends. The segments are randomly assembled using T4 DNA ligase and cloned into a yeast expression vector containing a minimal TATA box region and a reporter gene. The vector library of reassembled variants is transformed into a yeast strain that expresses the AlcR protein from an integrated DNA element.

Colonies are screened for expression of the reporter gene. Colonies with greater reporter expression than the progenitor AlcA promoter-reporter control strain are further characterized to quantify the level of promoter improvement.

### Example 5

Multiple promoter “assembly” of the Arabidopsis elongation factor 1A (EF-1A), ubiquitin 3 (UBQ-3), and protein kinase 1 (ATPK1) promoters is carried out to identify variants with higher expression levels than any of the progenitor molecules.

Approximately 1000-base pair regions of the EF-1A, UBQ-3, and ATPK1 promoters are amplified by the polymerase chain reaction from Arabidopsis thaliana genomic DNA. The cloned PCR products are cleaved into random segments using time-limited DNase I digestion. The segments are randomly assembled using T4 DNA ligase and cloned into a plant expression vector containing a minimal TATA box region and a GUS reporter gene. The vector library of reassembled variants is transformed into an Agrobacterium host that will allow gene transfer into plant cells.

Tobacco or Arabidopsis suspension cells are aliquoted into a 48-well microtiter plate and each well is infected with a unique Agrobacterium strain containing one reassembled variant. After 48 hours, reporter gene expression is determined in each well by histochemical staining with the beta-glucuronidase (GUS) substrate, X-GLUC. Cells/wells with greater color intensity than the progenitor promoters tested singly represent variants with potentially improved promoters and are referenced back to the appropriate Agrobacterium strain. Agrobacterium strains containing potentially improved promoter vectors are used to transform suspension cells or whole plants and the resulting cells characterized by enzymatic assays to quantify the level of promoter improvement.

### Example 6

Single promoter “assembly” of the Brassica napin (NapA) promoter is carried out to identify variants with altered developmental expression.

An approximately 900-base pair region of the NapA promoter is amplified by the polymerase chain reaction from Brassica napus genomic DNA. The cloned PCR product is cleaved into random segments using time-limited DNase I digestion. The segments are randomly assembled using T4 DNA ligase and cloned into a plant expression vector containing a minimal TATA box region and a GUS reporter gene.

The vector library of reassembled variants is transformed into an Agrobacterium host that will allow gene transfer into plant cells. Transgenic Brassica or Arabidopsis plants are generated by Agrobacterium-mediated transformation. Seeds at different stages of development are collected from individual transgenic plants and stained with the beta-glucuronidase (GUS) substrate, X-GLUC. Seeds in which the staining pattern for the napin promoter appears to be altered developmentally (for example, very high expression in early embryos) potentially contain interesting promoter variants. The promoter variants giving potentially interesting expression patterns can be isolated from the plant tissue by PCR, re-cloned into an expression vector, and their properties confirmed by an additional round of plant transformation.

### Example 7

Multiple promoter “assembly” of the Brassica A9 and Bnm1 promoters is carried out to identify variants with altered spatial expression patterns.

Approximately 1000-base pair regions of the A9 and Bnm1 promoters are amplified by the polymerase chain reaction from Brassica napus genomic DNA. The cloned PCR products are cleaved into random segments by mechanical shearing. The DNA samples are then end-repaired prior to ligation into a blunt-ended vector using a combination of T4 DNA polymerase, Klenow DNA polymerase, and T4 polynucleotide kinase. The segments are randomly assembled using T4 DNA ligase and cloned into a plant expression vector containing a minimal TATA box region and a GUS reporter gene.

The vector library of reassembled variants is transformed into an Agrobacterium host that will allow gene transfer into plant cells. Transgenic Brassica or Arabidopsis plants are generated by Agrobacterium-mediated transformation. Flowers at different stages of development are collected from individual transgenic plants and stained with the beta-glucuronidase (GUS) substrate, X-GLUC. Flowers in which the staining pattern appears to be altered spatially relative to the progenitor promoters tested individually (for example, expression in both pollen and tapetal cells) potentially contain interesting promoter variants. The promoter variants giving potentially interesting expression patterns can be isolated from the plant tissue by PCR, re-cloned into an expression vector, and their properties confirmed by an additional round of plant transformation.

### Example 8

Single promoter “assembly” of the strawberry vein-banding virus ³⁵S-like (SVBV) promoter is carried out to identify variants with higher expression levels in plant cells.

An approximately 475-base pair region of the “CaMV 35S-like” promoter (e.g., SEQ ID NO:1) is amplified by the polymerase chain reaction from strawberry vein-banding virus (SVBV) genomic DNA. The amplification process is carried out in the presence of a dNTP mixture that includes dUTP at a certain ratio relative to dTTP (the ratio can be altered to increase uracil incorporation and decrease the size of promoter fragments to be assembled). The PCR product is treated with uracil N-glycosylase and endonuclease IV to create single strand breaks at apurinic sites. Heat and alkali treatment can be used to remove the 2′-deoxyribose-5′-phosphate termini. DNA polymerase and polynucleotide kinase are used for strand displacement, extension, and end repair.

The vector library of reassembled variants is transformed into an Agrobacterium host that will allow gene transfer into plant cells. Transgenic Brassica or Arabidopsis plants are generated by Agrobacterium-mediated transformation. Flowers or other tissues at different stages of development are collected from individual transgenic plants and stained with the beta-glucuronidase (GUS) substrate, X-GLUC. Tissues in which the staining pattern appears to be altered spatially relative to the progenitor promoters tested individually potentially contain interesting promoter variants. The promoter variants giving potentially interesting expression patterns can be isolated from the plant tissue by PCR, re-cloned into an expression vector, and their properties confirmed by an additional round of plant transformation.

### Example 9

Single promoter “assembly” of the strawberry vein-banding virus 35S-like (SVBV) promoter is carried out to identify variants with higher expression levels in plant cells.

An approximately 475-base pair region of the “CaMV 35S-like” promoter (e.g., SEQ ID NO:1) is amplified by the polymerase chain reaction from strawberry vein-banding virus (SVBV) genomic DNA. The PCR product is cleaved into random segments using CviTI* restriction endonuclease under relaxed conditions (Megabase Research Products).

The segments are randomly assembled using T4 DNA ligase and size-selected for products greater than 200-base pairs in length by gel fractionation and purification. A double-stranded oligonucleotide tag containing 15-base pairs and including an AscI restriction site is ligated to the ends of the size-selected DNAs. PCR is then used to amplify the assembled products having the attached oligo, using a primer that is complementary to the oligo tag sequence. The PCR products are then cut with AscI and cloned into the compatible restriction site of a plant expression vector containing a minimal TATA box region and a GUS reporter gene.

The vector library of reassembled variants is transformed into an Agrobacterium host that will allow gene transfer into plant cells. Transgenic Brassica or Arabidopsis plants are generated by Agrobacterium-mediated transformation. Flowers or other tissues at different stages of development are collected from individual transgenic plants and stained with the beta-glucuronidase (GUS) substrate, X-GLUC. Tissues in which the staining pattern appears to be altered spatially relative to the progenitor promoters tested individually potentially contain interesting promoter variants. The promoter variants giving potentially interesting expression patterns can be isolated from the plant tissue by PCR, re-cloned into an expression vector, and their properties confirmed by an additional round of plant transformation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of reassembling polynucleotides involved in transcription, the method comprising,
providing a plurality of random polynucleotide segments from one or more transcriptional regulatory progenitor polynucleotides;
assembling the plurality of segments in a random fashion, thereby forming a plurality of reassembled polynucleotide; and
selecting a reassembled polynucleotide with a different transcriptional regulatory activity than the progenitor polynucleotides.

2. The method of claim 1, wherein the segments are from 5 bp to 5,000 bp long.

3. The method of claim 1, wherein the segments are less than 50 base pairs.

4. The method of claim 1, wherein the segments are greater than 49 base pairs.

5. The method of claim 1, wherein the assembling step comprises ligating the segments.

6. The method of claim 5, wherein the ligating step is performed by with a DNA ligase or a topoisomerase.

7. The method of claim 1, wherein the plurality of random segments comprises segments from at least two distinct promoter or enhancer polynucleotides.

8. The method of claim 1, wherein the plurality of random polynucleotide segments are obtained by random cleavage of one or more transcriptional regulatory progenitor polynucleotides.

9. The method of claim 1, wherein the plurality of random polynucleotide segments are obtained by random amplification of one or more part of one or more transcriptional regulatory progenitor polynucleotides.

10. The method of claim 1, wherein the reassembled polynucleotide comprises a promoter.

11. The method of claim 1, wherein the reassembled polynucleotide comprises an enhancer.

12. The method of claim 1, wherein the selection step comprises selecting a reassembled polynucleotide with increased transcriptional activity relative to the transcriptional activity of a progenitor polynucleotide.

13. The method of claim 1, wherein the selection step comprises selecting a reassembled polynucleotide with decreased transcriptional activity relative to the transcriptional activity of a progenitor polynucleotide.

14. The method of claim 1, wherein the selection step comprises selecting a reassembled polynucleotide with significant transcriptional activity in at least one cell or tissue type where the progenitor polynucleotide lacks activity.

15. The method of claim 1, wherein the selection step comprises selecting a reassembled polynucleotide without significant transcriptional activity in at least one cell or tissue type where the progenitor polynucleotide has activity.

16. The method of claim 1, wherein the selection step comprises selecting a reassembled polynucleotide with transcriptional activity that is activated in response to biotic or abiotic stimuli.

17. The method of claim 1, where the segments are formed by nicking and subsequent end-repair of DNA that is altered by radiation, oxidation, or a chemical agent.

18. The method of claim 1, wherein the selection step comprises selecting a reassembled polynucleotide with transcriptional activity at a different developmental stage of an organism relative to the transcriptional activity of a progenitor polynucleotide.

19. The method of claim 1, wherein the segments are formed by cleaving one or more progenitor polynucleotides with a restriction endonuclease.

20. The method of claim 1, wherein the segments are formed by cleaving one or more progenitor polynucleotides with DNaseI.

21. The method of claim 1, wherein the segments are formed by cleaving one or more progenitor polynucleotides mechanically.

22. The method of claim 1, wherein the segments are formed in a thermocyclic amplification reaction.

23. The method of claim 22, wherein the thermocyclic reaction is a polymerase chain reaction.

24. The method of claim 23, wherein the polymerase chain reaction is a mutagenic polymerase chain reaction.

25. The method of claim 1, wherein the selection step is performed by ligating the reassembled polynucleotide to a reporter gene and measuring reporter gene activity.

26. The method of claim 1, wherein the plurality of segments further comprises oligonucleotides.

27. The method of claim 26, wherein the oligonucleotide sequence corresponds to a transcription factor binding site.

28. The method of claim 26, wherein the nucleotide sequence of the oligonucleotides are not from a transcriptional regulatory polynucleotide.

29. The method of claim 1, wherein the reassembled polynucleotide is shorter than the progenitor polynucleotide.

30. The method of claim 1, wherein the reassembled polynucleotide is longer than the progenitor polynucleotide.

31. The method of claim 1, wherein the progenitor polynucleotides comprise allelic variants of a transcriptional regulator polynucleotide.

32. The method of claim 1, wherein the progenitor polynucleotides comprise plant transcriptional regulatory polynucleotides.

33. The method of claim 1, wherein the progenitor polynucleotides comprise yeast transcriptional regulatory polynucleotides.

34. The method of claim 1, wherein the progenitor polynucleotides comprise fungal transcriptional regulatory polynucleotides.

35. The method of claim 1, wherein the progenitor polynucleotides comprise mammalian transcriptional regulatory polynucleotides.

36. The method of claim 1, wherein the progenitor polynucleotides comprise viral transcriptional regulatory polynucleotides.

37. The method of claim 1, wherein the progenitor polynucleotides comprise bacterial transcriptional regulatory polynucleotides.

38. The method of claim 1, wherein the progenitor polynucleotides consist of one transcriptional regulatory polynucleotide.

39. The method of claim 1, wherein the transcriptional regulatory progenitor polynucleotides comprise more than one transcriptional regulatory polynucleotide.

40. The method of claim 1, wherein the transcriptional regulatory progenitor polynucleotides are less than 70% identical.

41. The method of claim 1, wherein the progenitor polynucleotides are less than 50% identical.

42. The method of claim 1, wherein the progenitor polynucleotides do not hybridize to each other following at least one wash in 0.2×SSC at 55° C. for 20 minutes.

43. The method of claim 1, wherein the polynucleotide segments are single stranded.

44. The method of claim 1, wherein the polynucleotide segments are double-stranded.

45. The method of claim 44, wherein the double-stranded segments have at least one overhanging single-stranded end.

46. The method of claim 45, wherein the overhanging single-stranded end comprises fewer than 10 base pairs.

47. The method of claim 1, wherein the assembling step does not comprise a polymerase.

48. A reassembled polynucleotide of claim 1.
